(19) United States
(12) Patent Application Publication
(10) Pub. No.: US 2024/0394955 A1
(43) Pub. Date: Nov. 28, 2024

(54) System and Method for Rendering Images

(71) Applicant: Microsoft Technology Licensing, LLC, Redmond, WA (US)

(72) Inventors: Risto Fermin Rangel Kuoppa, Woodinville, WA (US); Ivan Tarapov, Redmond, WA (US)

(21) Appl. No.: 18/321,561

(22) Filed: May 22, 2023

(51) Int. Cl. G06T 15/06; G06T 1/20; G06T 15/80; G16H 30/20

(52) U.S. Cl. CPC G06T 15/06; G06T 1/20; G06T 15/80; G16H 30/20

(57) ABSTRACT

A method, computer program product, and computing system for receiving a three-dimensional image data set for processing on a medical imaging system; processing the three-dimensional image data set on a central processing unit to generate a CPU output; processing the three-dimensional image data set on a graphical processing unit, if the graphical processing unit is available, to calculate a segmentation volume surface normal and generate a GPU output; and combining the CPU output and the GPU output to generate a rendered three-dimensional medical volume that depicts various tissue densities; wherein the GPU output includes a plurality of rendered frames.

### TECHNICAL FIELD

This disclosure relates to systems and methods for rendering images and, more particularly, to systems and methods for rendering images using a GPU.

### BACKGROUND

Medical images are often generated from voluminous medical volume point cloud data sets. Such medical volume point cloud data sets may be generated using MRI (i.e., Magnetic Resonance Imaging) systems and CAT (i.e., Computerized Axial Tomography) systems.

When rendering three-dimensional images from these medical volume point cloud data sets, the processing times may be prohibitively long due to the amount of computational horsepower required. Further, the utilization of microprocessors that have a low level of parallelism (e.g., that simultaneously execute a low quantity of threads) may result in a low level of performance. For example and when utilizing such low-parallelism systems, rendering times of upward of seven minutes may be realized when rendering a 512×512×800 voxel three-dimensional space.

### BRIEF DESCRIPTION OF THE DRAWINGS

FIG. 1 is a flow chart of one implementation of a volume rendering process according to an embodiment of the present disclosure;

FIG. 2 is a diagrammatic view of a hybrid architecture platform that effectuates the volume rendering process of FIG. 1 according to an embodiment of the present disclosure; and

FIGS. 3-5 are diagrammatic views of rendered three-dimensional medical volumes generated by the volume rendering process of FIG. 1.

Like reference symbols in the various drawings indicate like elements.

### DETAILED DESCRIPTION OF THE PREFERRED EMBODIMENTS

As will be discussed below in greater detail, implementations of the present disclosure are configured to generate interactive real-time 3D renderings of medical volumes and medical volume segmentations. The system is a combination of linear and parallel computing components, wherein some components execute on a CPU and others execute on GPU, thus leveraging the massively parallel architecture of today's GPUs to greatly enhance system performance.

The details of one or more implementations are set forth in the accompanying drawings and the description below. Other features and advantages will become apparent from the description, the drawings, and the claims.

### Volume Rendering Process:

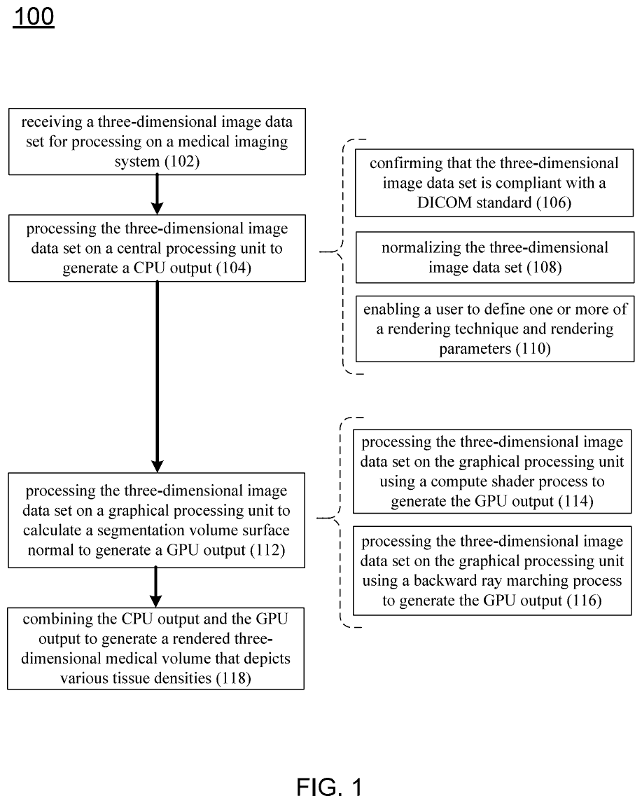

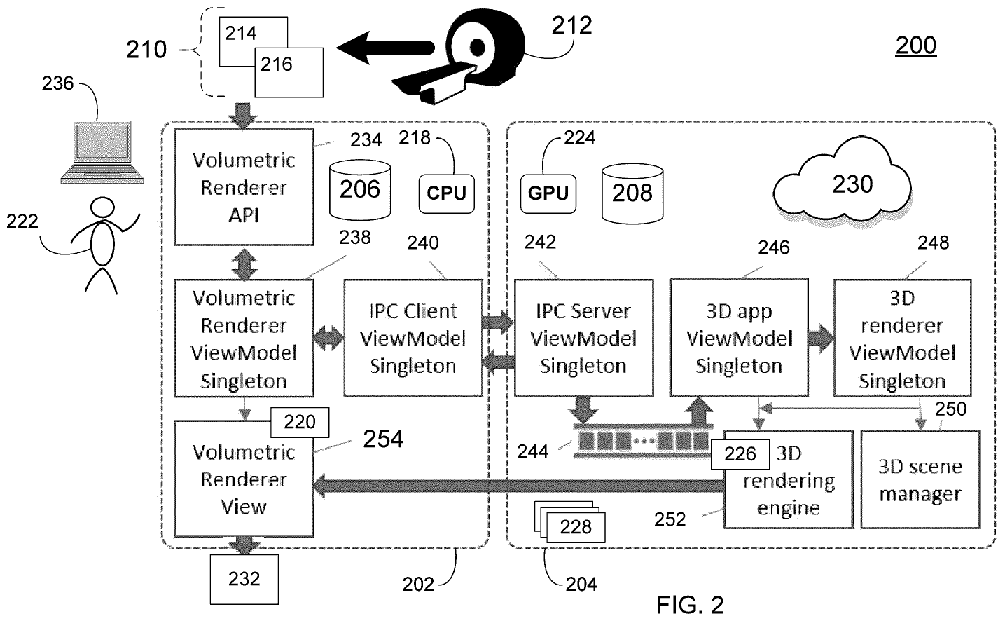

Referring to FIGS. 1-2, there is shown volume rendering process 100, which may be executed on a hybrid architecture platform 200 that includes both a CPU architecture portion (e.g., CPU portion 202) and a GPU architecture portion (e.g., GPU portion 204). CPU portion 202 may include a CPU (i.e., central processing unit) that is configured to process data, while GPU portion 204 may include a GPU (i.e., graphical processing unit) that is configured to render images. Most of CPU portion 202 components execute exclusively on a CPU. And while some of GPU portion 204 components can execute or interact with the GPU, not all of GPU portion 204 components strictly execute on a GPU

A Central Processing Unit (CPU) is the primary component of a computer that performs most of the processing and calculations required to run programs and operate the computer. The CPU is responsible for executing instructions and controlling the operation of the computer and reads instructions from memory, interprets them, and then executes them to perform calculations, manipulate data, and control input/output operations. The CPU is composed of several components, including the arithmetic logic unit (ALU), the control unit (CU), and the cache. The ALU performs arithmetic and logical operations, while the CU controls the flow of instructions and data between the CPU and memory. The cache is a portion of high-speed memory that stores frequently accessed data and instructions to improve performance. Modern CPUs can have multiple cores, which are essentially separate CPUs on the same chip, which allows them to perform multiple tasks simultaneously, improving performance and efficiency.

A Graphical Processing Unit (GPU) is a specialized processor designed to handle the complex calculations required for graphics rendering. GPUs are highly parallel, meaning that they can perform many calculations simultaneously, making them ideal for the types of calculations required for graphics processing. GPUs were originally developed for use in computer graphics and gaming applications, but they are now used in a variety of fields, including scientific computing, machine learning, and cryptocurrency mining. This has led to the development of specialized GPU architectures, such as Nvidia's CUDA architecture, which is designed specifically for parallel computing tasks. GPUs typically have many small processing cores, compared to the fewer, but more powerful cores found in a CPU. This allows the GPU to perform many calculations simultaneously, improving performance and efficiency. GPUs also have specialized memory architectures that are optimized for the types of calculations required for graphics processing.

The instruction sets and subroutines of volume rendering process 100, which may be stored on storage device 206 coupled to CPU portion 202 and/or storage device 208 coupled to GPU portion 204, may be executed by one or more processors (not shown) and one or more memory architectures (not shown) incorporated into CPU portion 202 and/or GPU portion 204. Storage devices 206, 208 may include but are not limited to: hard disk drives; optical drives; RAID devices; random access memories (RAM); read-only memories (ROM), and all forms of flash memory storage devices. Additionally/alternatively, volume rendering process 100 may be implemented as an entirely hardware embodiment, an entirely software embodiment (including firmware, resident software, micro-code, etc.) or an embodiment combining software and hardware aspects that may all generally be referred to herein as a “circuit,” “module”. “process” or “system”. Accordingly, such implementation flexibility allows volume rendering to occur on a variety of systems, even those that lack a dedicated GPU module by switching the computations to CPU automatically”.

Volume rendering process 100 may receive 102 a three-dimensional image data set (e.g., three-dimensional image data set 210) for processing on a medical imaging system (e.g., hybrid architecture platform 200), wherein three-dimensional image data set 210 may consist of a three-dimensional point cloud in which each point corresponds to a density value.

In some implementations, this three-dimensional image data set (e.g., three-dimensional image data set 210) includes a three-dimensional image data set (e.g., three-dimensional image data set 210) generated via a medical imaging system (e.g., medical imaging system 212), examples of which may include but are not limited to an MRI system and/or a CAT system.

A Magnetic Resonance Imaging (MRI) system is a medical imaging device that uses a strong magnetic field and radio waves to generate detailed images of the body's internal structures. The MRI system consists of a large magnet, a radiofrequency (RF) coil, and a computer system that controls the image acquisition process. When a patient is placed inside the MRI machine, the strong magnetic field aligns the protons in the body's tissues. The RF coil then emits radio waves that cause the protons to resonate, creating a detectable signal that is used to generate an image. MRI is a non-invasive imaging technique that can be used to examine many different parts of the body, including the brain, spinal cord, joints, and organs. It is particularly useful for examining soft tissues, such as muscles, tendons, and ligaments, and can be used to diagnose a wide range of conditions, including tumors, infections, and injuries. MRI is generally considered safe, as it does not use ionizing radiation, but patients with certain types of medical implants, such as pacemakers, may not be able to undergo an MRI due to potential safety risks.

A Computerized Axial Tomography (CAT) system, also known as a Computed Tomography (CT) scanner, is a medical imaging device that uses X-rays to create detailed, cross-sectional images of the body. The CAT system consists of a large, doughnut-shaped machine that houses an X-ray tube and a detector. When a patient is placed inside the CT scanner, the X-ray tube rotates around the body, emitting a series of narrow X-ray beams through the body at different angles. The detector then measures the amount of radiation that passes through the body and creates a detailed image of the internal structures. CT scans are particularly useful for examining internal organs and structures, such as the brain, chest, abdomen, and pelvis. They can be used to diagnose a wide range of conditions, including tumors, infections, and injuries. Modern CT scanners can produce highly detailed images with very low radiation doses, making them a safe and effective diagnostic tool.

In some implementations, this three-dimensional image data set (e.g., three-dimensional image data set 210) includes a medical volume point cloud data set (e.g., medical volume point cloud data set 214). A medical volume point cloud refers to a three-dimensional representation of a medical image dataset that is composed of a large number of individual points in space, each with its own unique set of coordinates and intensity values. This type of point cloud is typically generated using specialized imaging techniques such as computed tomography (CT), magnetic resonance imaging (MRI), or ultrasound, which allow for the acquisition of high-resolution, three-dimensional medical images. Medical volume point clouds are useful in a variety of applications, such as medical diagnosis, treatment planning, and research. They can be used to create detailed 3D visualizations of internal structures, such as organs, bones, or tumors, that can help doctors and researchers better understand and analyze complex medical data. Additionally, medical volume point clouds can be used to create physical models of patient anatomy, which can be used for surgical planning and simulation.

Additionally/alternatively and in some implementations, this three-dimensional image data set includes: a medical volume segmentations point cloud data set (e.g., medical volume segmentations point cloud data set 216). A medical volume segmentation point cloud is a type of three-dimensional medical image representation that is generated by segmenting a medical volume point cloud (e.g., medical volume point cloud data set 214) into distinct regions or structures of interest. In other words, the segmentation process involves identifying and separating different parts of the original point cloud based on specific criteria, such as intensity values, shape, or texture. The resulting segmented point cloud consists of a set of individual points, each of which is labeled with a unique identifier that corresponds to a particular anatomical structure or region of interest. This type of point cloud can be used to create detailed 3D visualizations of specific structures, such as blood vessels, tumors, or other lesions, that can be helpful in medical diagnosis, treatment planning, and research. Medical volume segmentation point clouds are widely used in a variety of medical applications, including radiation therapy planning, surgical simulation and planning, and drug development. They can also be used to create physical models of patient anatomy for use in surgical training or other educational applications.

The medical volume segmentations point cloud data set (e.g., medical volume segmentations point cloud data set 216) may be generated via machine and/or human processing of the medical volume point cloud data set (e.g., medical volume point cloud data set 214).

Volume rendering process 100 may process 104 the three-dimensional image data set (e.g., three-dimensional image data set 210) on a central processing unit (e.g., CPU 218 within CPU portion 202) to generate a CPU output (e.g., CPU output 220).

When processing 104 the three-dimensional image data set (e.g., three-dimensional image data set 210) on a central processing unit (e.g., CPU 218 within CPU portion 202) to generate a CPU output (e.g., CPU output 220), volume rendering process 100 may confirm 106 that the three-dimensional image data set (e.g., three-dimensional image data set 210) is compliant with a DICOM standard.

The DICOM standard (Digital Imaging and Communications in Medicine) is a widely used set of rules and protocols for the storage, transmission, and communication of medical images and related data. The standard was developed by the National Electrical Manufacturers Association (NEMA) and the American College of Radiology (ACR) and is currently maintained by the DICOM Standards Committee. DICOM provides a standard format for the exchange of medical images and related data between different medical devices and systems, such as scanners, PACS (Picture Archiving and Communication System) workstations, and printers. The standard defines a common language for medical imaging that ensures interoperability and compatibility between different systems, regardless of the manufacturer or the technology used. DICOM images typically contain a combination of visual and non-visual information, including patient demographics, study information, and image data. The standard supports a wide range of image modalities, including CT (Computed Tomography), MRI (Magnetic Resonance Imaging), X-ray, and ultrasound, among others. Overall, the DICOM standard is critical for the efficient and effective exchange of medical imaging information and plays a central role in modern medical imaging and healthcare.

Further and when processing 104 the three-dimensional image data set (e.g., three-dimensional image data set 210) on a central processing unit (e.g., CPU 218 within CPU portion 202) to generate a CPU output (e.g., CPU output 220), volume rendering process 100 may normalize 108 the three-dimensional image data set (e.g., three-dimensional image data set 210) to e.g., maximize the differences between regions of different densities defined within the three-dimensional image data set (e.g., three-dimensional image data set 210).

Normalizing data is the process of transforming numerical data into a standardized format to eliminate any inherent bias or scale differences that may exist in the data. The goal of normalization is to ensure that all variables are on a similar scale so that they can be compared and analyzed together more effectively. Normalization can be done in various ways, but one common method is to rescale the data to have a mean of zero and a standard deviation of one. This process is called standardization or Z-score normalization. It involves subtracting the mean of the data from each value and then dividing by the standard deviation. Another common normalization technique is called min-max scaling, which rescales the data to a fixed range between 0 and 1. This is done by subtracting the minimum value from each value and then dividing by the range of the data (i.e., the maximum value minus the minimum value). Normalization is often used in machine learning and data analysis to improve the performance and accuracy of algorithms that rely on numerical data. By normalizing the data, it is easier to compare different variables and identify patterns and relationships between them.

For example and when normalizing 108 the three-dimensional image data set (e.g., three-dimensional image data set 210), volume rendering process 100 may rescale and/or rebase the three-dimensional image data set (e.g., three-dimensional image data set 210) to ensure that volume rendering process 100 may properly generate an interactive real-time 3D rendering.

Rescaling data concerns transforming the original numerical data to a different range or scale. The goal of rescaling is often to bring the data into a more manageable or meaningful range for analysis or visualization. Another common technique transforms the data to a fixed range between 0 and 1, with the minimum value becoming 0 and the maximum value becoming 1. Rescaling can also be used to convert data between different units of measurement. For example, if data is originally recorded in pounds, it may be rescaled to kilograms for analysis. Rescaling can be important in many areas of data analysis, including machine learning, where algorithms may perform better on data that is within a certain range or has a standardized scale. Additionally, rescaling can make it easier to compare different variables or datasets that may have different units or scales.

Rebasing data refers to the process of changing the base value of a dataset or index to a different starting point. This is commonly used in finance and economics to track changes in a variable over time. For example, if a stock index is originally set to 100 on January 1st, but then undergoes significant changes in value over the course of the year, it may be rebased to a new starting point (e.g., 200) to more accurately reflect the changes in value. Rebasing can be done using a variety of techniques, but the most common method is to multiply each data point by a constant factor that adjusts the base value to the new starting point. This ensures that the relative changes in value remain the same, even though the base value has been shifted. Rebasing can be important in comparing different time periods or datasets that have different base values, and it can help to reveal trends and patterns that may not be immediately apparent when using the original base value.

Additionally and when processing 104 the three-dimensional image data set (e.g., three-dimensional image data set 210) on a central processing unit (e.g., CPU 218 within CPU portion 202) to generate a CPU output (e.g., CPU output 220), volume rendering process 100 may enable 110 a user (e.g., user 222) to define one or more of a rendering technique/parameters for use when generating an interactive real-time 3D rendering.

For example, volume rendering process 100 may enable 110 the user (e.g., user 222) to select the rendering technique that they wish to have applied to the three-dimensional image data set (e.g., three-dimensional image data set 210). Examples may include but are not limited to various transfer functions that associate a specific color map to a range of densities, wherein a specific color is assigned to a voxel within three-dimensional image data set 210 based upon the density of the portion of the body being imaged by that voxel. For example, a voxel representing a portion of a heart may have a different density (and a different color) than a voxel representing a portion of a lung, which may have a different density (and a different color) than a voxel representing a portion of a bone, which may have a different density (and a different color) than a voxel representing a portion of a muscle.

A voxel is a three-dimensional unit of a digital image, similar to a two-dimensional pixel in a digital photograph. The term voxel is derived from the words “volume” and “pixel”. A voxel represents a single point in a three-dimensional space and contains information about its location in the image as well as its corresponding intensity or color value. In medical imaging, voxels are used to represent the smallest possible unit of a medical image, such as a CT (Computed Tomography) or MRI (Magnetic Resonance Imaging) scan. Each voxel represents a tiny cube of tissue or material within the image, and its intensity value reflects the physical properties of that particular cube. Voxels are essential for creating three-dimensional reconstructions of medical images, which can be used for diagnosis, treatment planning, and medical research. By analyzing the intensity and location of individual voxels within an image, medical professionals can identify specific structures or abnormalities within the body and develop targeted treatment plans. Overall, voxels are a fundamental component of modern medical imaging technology, allowing for the accurate and detailed representation of the human body in three dimensions.

Volume rendering process 100 may process 112 the three-dimensional image data set (e.g., three-dimensional image data set 210) on a graphical processing unit (e.g., GPU 224 within GPU portion 204), if a graphical processing unit (e.g., GPU 224 within GPU portion 204) is available, to calculate a segmentation volume surface normal and generate a GPU output (e.g., GPU output 226). The GPU output (e.g., GPU output 226) includes a plurality of rendered frames (e.g., plurality of rendered frames 228). In the event that such a graphical processing unit (e.g., GPU 224 within GPU portion 204) is not available, volume rendering process 100 may process the three-dimensional image data set (e.g., three-dimensional image data set 210) on the central processing unit (e.g., CPU 218 within CPU portion 202) in it entirety.

A segmentation volume surface normal is a type of geometric property that can be computed from a three-dimensional medical image segmentation (e.g., medical volume segmentations point cloud data set 216). In segmentation, the image is divided into distinct regions based on specific criteria, such as intensity values, shape, or texture. Once a segmentation has been generated, a surface normal can be computed for each point on the surface of the segmented region. The surface normal is a vector that is perpendicular to the tangent plane at the given point on the surface. It provides information about the orientation and direction of the surface at each point, and can be used to estimate lighting and shading effects when rendering the surface. The segmentation volume surface normal is important in medical imaging because it can be used to generate realistic and accurate 3D visualizations of segmented regions, such as organs, tumors, or other structures of interest. The surface normal information can be used to create smooth and visually appealing surface renderings that accurately represent the shape and orientation of the segmented region. The segmentation volume surface normal can also be used for other applications, such as volume reconstruction, deformable image registration, and image-guided interventions.

In some implementation of volume rendering process 100, the graphical processing unit (e.g., GPU 224 within GPU portion 204) can be but is not limited to a local graphical processing unit. In other implementation of volume rendering process 100, the graphical processing unit (e.g., GPU 224 within GPU portion 204) can be but is not limited to a cloud-based graphical processing unit. For example, some or all of the GPU architecture portion (e.g., GPU portion 204) may be a cloud-based resource (e.g., cloud-based resource 230), thus enabling volume rendering process 100 to access and utilize the vast computation resources and massive parallelism of such a cloud-based resource.

Parallelism in computing refers to the ability of a computer system to simultaneously execute multiple tasks or processes in parallel, typically by using multiple processors or cores. In a parallel computing system, multiple processors or cores work together to perform a task, dividing the work into smaller sub-tasks that can be executed in parallel. Parallelism can provide significant performance benefits by allowing tasks to be completed more quickly than they would be in a serial computing system, where tasks are executed one after the other. Parallelism can be achieved using various techniques, such as multi-threading, multi-processing, and distributed computing.
Multi-Threading involves dividing a single task into multiple threads of execution, each running on a separate processor or core. Each thread can execute independently, allowing multiple tasks to be executed in parallel.
Multi-Processing involves using multiple processors or cores to execute multiple tasks simultaneously. Each processor or core can execute its own set of instructions, allowing multiple tasks to be executed in parallel.
Distributed Computing involves using multiple computers or servers to work together to execute a task. Each computer or server can work on a different portion of the task, allowing large tasks to be completed more quickly than would be possible on a single computer.

When processing 112 the three-dimensional image data set (e.g., three-dimensional image data set 210) on a graphical processing unit (e.g., GPU 224 within GPU portion 204) to calculate a segmentation volume surface normal and generate a GPU output (e.g., GPU output 226), volume rendering process 100 may process 114 the three-dimensional image data set (e.g., three-dimensional image data set 210) on the graphical processing unit (e.g., GPU 224 within GPU portion 204) using one or more compute shader processes and one or more graphics shaders processes to generate the GPU output (e.g., GPU output 226).

A compute shader is a type of shader used in modern graphics processing units (e.g., GPU 224 within GPU portion 204) to perform general-purpose computations. Unlike traditional graphics shaders, which are designed to manipulate vertices and pixels in a scene, compute shaders are specifically designed for parallel computation tasks, such as numerical simulations, scientific calculations, or data processing. Compute shaders are programmable units of the graphical processing unit (e.g., GPU 224 within GPU portion 204) that can be used to perform massively parallel calculations on large sets of data (e.g., three-dimensional image data set 210), which allow developers to offload computationally intensive tasks from the CPU (e.g., CPU 218 within CPU portion 202) to the GPU (e.g., GPU 224 within GPU portion 204), taking advantage of the GPU's parallel processing power to accelerate the computation. Compute shaders operate on data organized in buffers, which are typically stored in the GPU's memory, wherein these buffers can be accessed and manipulated by the compute shader, thus allowing for efficient computation on large sets of data. Compute shaders are widely used in scientific simulations, machine learning, and other applications that require high-performance computing, in that they offer a flexible and efficient way to perform large-scale computations using the parallel processing power of the GPU.

Additionally and when processing 112 the three-dimensional image data set (e.g., three-dimensional image data set 210) on a graphical processing unit (e.g., GPU 224 within GPU portion 204) to calculate a segmentation volume surface normal and generate a GPU output (e.g., GPU output 226), volume rendering process 100 may process 116 the three-dimensional image data set (e.g., three-dimensional image data set 210) on the graphical processing unit (e.g., GPU 224 within GPU portion 204) using a backward ray marching process to generate the GPU output (e.g., GPU output 226).

A backward ray marching algorithm is a computer graphics technique used for rendering three-dimensional volumetric data, such as medical images or scientific simulations. It is a technique for visualizing the interior of a three-dimensional volume by tracing rays of light backwards through the volume, starting from the viewer's eye and moving towards the light source. The backward ray marching algorithm works by casting a ray of light from each pixel on the image plane, through the volume, towards the viewer. As the ray passes through the volume, it accumulates the colors and opacity values of the voxels it encounters, generating a color and opacity value for the pixel. The ray continues to march backwards until it either exits the volume or reaches the light source. To calculate the color and opacity values for each pixel, the algorithm uses a transfer function, which maps the intensity values of the voxels to color and opacity values. This transfer function can be adjusted to enhance or suppress certain features in the image. The backward ray marching algorithm is a popular technique for rendering volumetric data because it can produce high-quality images with accurate lighting and shading effects. It is particularly well-suited for medical imaging applications, where it is used to create detailed and realistic 3D visualizations of internal organs and structures.

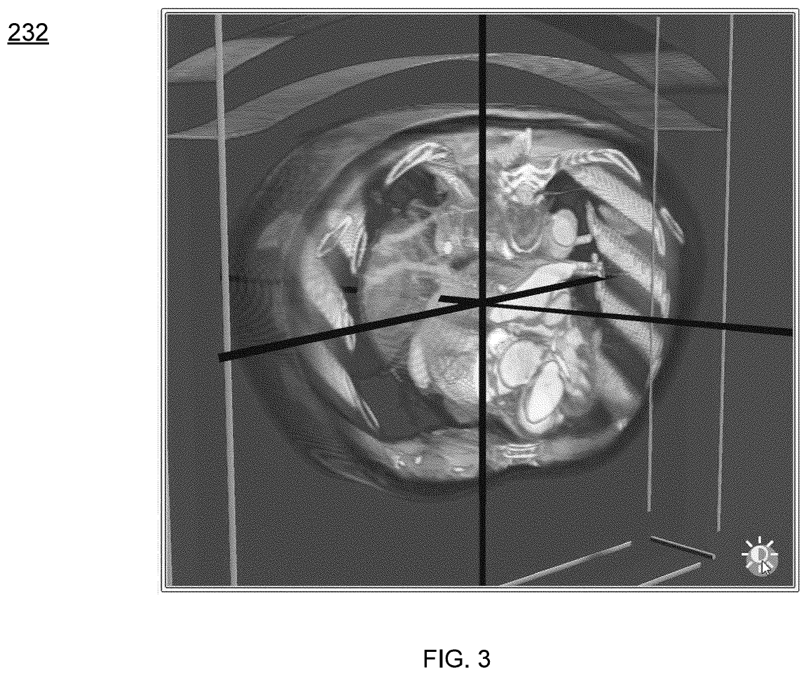

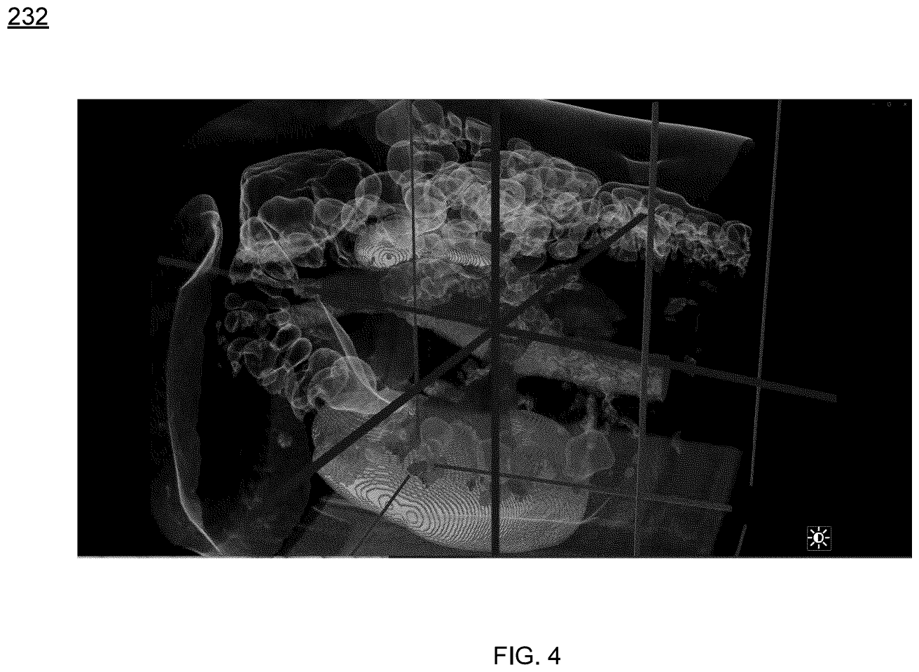

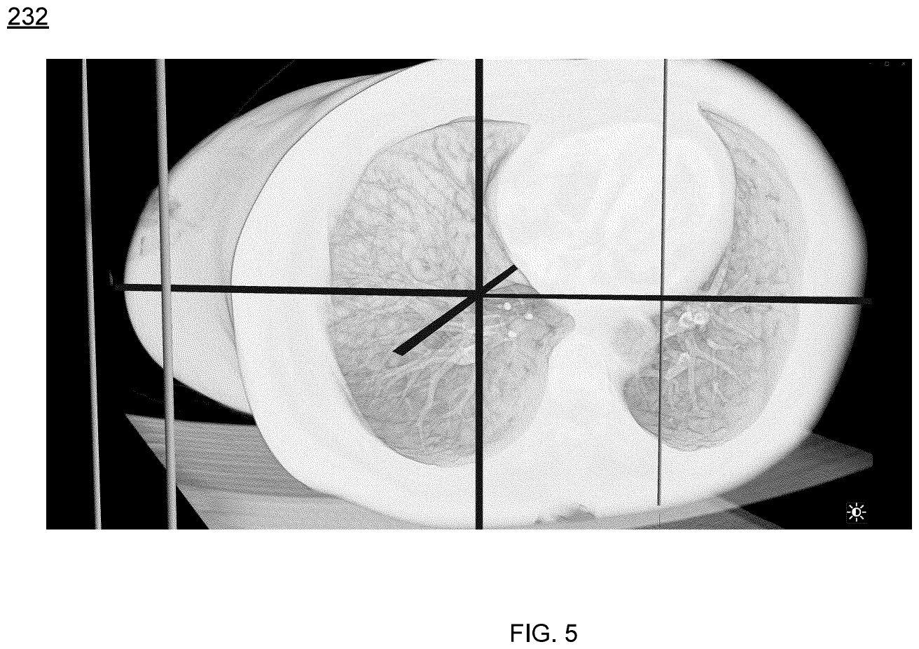

Volume rendering process 100 may combine 118 the CPU output (e.g., CPU output 220) and the GPU output (e.g., GPU output 226) to generate a rendered three-dimensional medical volume (e.g., rendered three-dimensional medical volume 232) that depicts various tissue densities. Referring also to FIGS. 3-5, there are shown various examples of rendered three-dimensional medical volume 232.

### Exemplary Implementations

CPU portion 202 may include volumetric render API 234 that is (generally speaking) an application program interface that exposes the functionality of volume rendering process 100 to a client (e.g., user 222 via client device 236), thus allowing the user (e.g., user 222) to effectuate the processing of the three-dimensional image data set (e.g., three-dimensional image data set 210) by volume rendering process 100 to generate the rendered three-dimensional medical volume (e.g., rendered three-dimensional medical volume 232). Examples of client device 236 may include, but are not limited to a personal digital assistant, a tablet computer, a laptop computer, a smart phone, a personal computer, a notebook computer, a server computer and a dedicated network device. Client device 236 may execute an operating system, examples of which may include but are not limited to Microsoft Windows™, Android™, iOS™, Linux™, or a custom operating system.

Volumetric render API 234 may allow the client (e.g., user 222 via client device 236) to define various options, examples of which may include but are not limited to: adding a slice to an image, removing a slice from an image, changing the color of a segmentation, starting the rendering of rendered three-dimensional medical volume 232, stopping/pausing the rendering of rendered three-dimensional medical volume 232, modifying the virtual camera position and orientation of rendered three-dimensional medical volume 232, modifying the scene rendering parameters of rendered three-dimensional medical volume 232, etc.

CPU portion 202 may include volumetric renderer singleton 238, which may define the logical state of rendering process and provide the same to the client (e.g., user 222 via client device 236) to let them know if volume rendering process 100 is e.g., currently rendering/transferring information to GPU portion 204/has completed rendering/etc.

CPU portion 202 may include IPC Client ViewModel Singleton 240, wherein GPU portion 204 may include IPC Server ViewModel Singleton 242 to enable InterProcess Communication between CPU portion 202 and GPU portion 204. Accordingly, the combination of IPC Client ViewModel Singleton 240 and IPC Server ViewModel Singleton 242 may enable e.g., the opening of ports, the registering of logical states, the providing of messages/instructions, the transfer of data (e.g., some or all of three-dimensional image data set 210). Generally speaking, the combination of IPC Client ViewModel Singleton 240 and IPC Server ViewModel Singleton 242 functions as the traffic cop of the communication channel between CPU portion 202 and GPU portion 204, as it acts like a communications manager.

In computer programming, a singleton is a design pattern that restricts the instantiation of a class to a single object. This means that, in a program, only one instance of a class can exist at any given time, and this instance can be accessed globally by any part of the program that needs it. The singleton pattern is often used to control access to shared resources or to ensure that certain operations are performed only once. For example, a singleton class could be used to manage a database connection, where it is important to ensure that there is only one connection to the database at any given time. Similarly, a singleton could be used to manage access to a hardware device, such as a printer or a network interface. In order to implement the singleton pattern, the class constructor is typically made private, which prevents other objects from creating instances of the class directly. Instead, a static method is provided that returns the single instance of the class, creating it if necessary. The class also typically contains a private static variable that holds the single instance of the class.

Interprocess communication (IPC) is a mechanism that allows different processes running on the same or different machines to communicate with each other. IPC is an essential component of modern operating systems and enables multiple processes to coordinate and share resources, leading to more efficient and scalable systems.

There are several types of IPC mechanisms, including:
Pipes: A pipe is a simple form of IPC that allows two processes to communicate with each other by reading and writing to a common buffer.
Shared Memory: Shared memory is a technique where multiple processes can access a common block of memory. This enables efficient data transfer between processes and is often used in high-performance computing applications.
Message Passing: In message passing, processes communicate by sending and receiving messages through a message queue or mailbox. This mechanism can be used for both synchronous and asynchronous communication.
Remote Procedure Call (RPC): RPC allows a process to execute a function or procedure in a remote process as if it were executing locally. This is often used in distributed systems, where processes are running on different machines.

IPC is used in a variety of applications, such as client-server architectures, distributed computing, and operating systems, wherein IPC enables processes to communicate with each other, share resources, and coordinate their activities; leading to more efficient and scalable systems.

As discussed above and in some implementations of volume rendering process 100, the graphical processing unit (e.g., GPU 224 within GPU portion 204) may be a cloud-based graphical processing unit, wherein some or all of the GPU architecture portion (e.g., GPU portion 204) may be a cloud-based resource (e.g., cloud-based resource 230). Accordingly, IPC Client ViewModel Singleton 240 and IPC Server ViewModel Singleton 242 may be configured to communicate across multiple communication mediums (e.g., local data buses, local area networks, wide area networks, the internet).

In some implementations, CPU portion 202 contains several components using the singleton pattern. However, there can be more than CPU portion 202 instantiations that communicate to the same GPU portion 204. The CPU portion 202 may be but is not limited to one single instantiation communicating to the same GPU portion instance.

GPU portion 204 may include communication queue 244 to temporarily store inbound data received from CPU portion 202 for subsequent processing by GPU portion 204, thus preventing conflicts & overwrites, data loss and poor communication performance.

GPU portion 204 may include 3D App ViewModel Singleton 246 that controls what is being seen by the client (e.g., user 222 via client device 236) in a user interface (not shown) that the client (e.g., user 222) is using to manipulate the rendered three-dimensional medical volume (e.g., rendered three-dimensional medical volume 232) via e.g., mouse movements, etc.

GPU portion 204 may include 3D Renderer ViewModel Singleton 248 that is a state manager for the rendered three-dimensional medical volume (e.g., rendered three-dimensional medical volume 232) to enable control of what segmentations are processed, which of those segmentations are rendered at the same time (e.g., lungs and heart), and what is being rendered in the user interface (not shown) that the client (e.g., user 222) is viewing.

GPU portion 204 may include three-dimensional scene manager 250 that defines e.g., textures for colors of rendered three-dimensional medical volume 232, the memory space for different layers of rendered three-dimensional medical volume 232, and the three-dimensional texture abstractions for rendered three-dimensional medical volume 232; thus basically defining the specifics and parameters of three-dimensional rendering engine 252.

GPU portion 204 may include three-dimensional rendering engine 252 that abstracts the GPU (e.g., GPU 224 within GPU portion 204) and provides instructions for how three-dimensional image data set 210 (which is stored within memory) is transferred to the graphical processing unit (e.g., GPU 224 within GPU portion 204) via e.g., DirectX, OpenGL, Vulcan, or some other methodology.

Three-dimensional rendering engine 252 may generate GPU output 226 (which includes plurality of rendered frames 228), which is provided to CPU portion 202 for combining 118 with CPU output 220 to generate the rendered three-dimensional medical volume (e.g., rendered three-dimensional medical volume 232). For example, each of the plurality of rendered frames 228 may be stored within GPU portion 204 at a defined memory address so that volume rendering process 100 may obtain the same and combine 118 CPU output 220 and GPU output 226 to generate rendered three-dimensional medical volume 232. For example, CPU portion 202 may include volumetric renderer 254, which may obtain each of the plurality of rendered frames 228 stored within GPU portion 204 and combine 118 the same with CPU output 220 to generate rendered three-dimensional medical volume 232.

### General

The present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module”, “process” or “system.” Furthermore, the present disclosure may take the form of a computer program product on a computer-usable storage medium having computer-usable program code embodied in the medium.

Any suitable computer usable or computer readable medium may be used. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object-oriented programming language. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet.

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general-purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, not at all, or in any combination with any other flowcharts depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
receiving a three-dimensional image data set for processing on a medical imaging system;
processing the three-dimensional image data set on a central processing unit to generate a CPU output;
processing the three-dimensional image data set on a graphical processing unit, if the graphical processing unit is available, to calculate a segmentation volume surface normal and generate a GPU output; and
combining the CPU output and the GPU output to generate a rendered three-dimensional medical volume that depicts various tissue densities;
wherein the GPU output includes a plurality of rendered frames.

2. The computer-implemented method of claim 1 wherein processing the three-dimensional image data set on a central processing unit to generate a CPU output includes:
confirming that the three-dimensional image data set is compliant with a DICOM standard.

3. The computer-implemented method of claim 1 wherein processing the three-dimensional image data set on a central processing unit to generate a CPU output includes:
normalizing the three-dimensional image data set.

4. The computer-implemented method of claim 1 wherein processing the three-dimensional image data set on a central processing unit to generate a CPU output includes:
enabling a user to define one or more of a rendering technique and rendering parameters.

5. The computer-implemented method of claim 1 wherein processing the three-dimensional image data set on a graphical processing unit to calculate a segmentation volume surface normal and generate a GPU output includes:
processing the three-dimensional image data set on the graphical processing unit using a compute shader process to generate the GPU output.

6. The computer-implemented method of claim 1 wherein processing the three-dimensional image data set on a graphical processing unit to calculate a segmentation volume surface normal and generate a GPU output includes:
processing the three-dimensional image data set on the graphical processing unit using a backward ray marching process to generate the GPU output.

7. The computer-implemented method of claim 1 wherein the three-dimensional image data set includes:
a medical volume point cloud data set.

8. The computer-implemented method of claim 1 wherein the three-dimensional image data set includes one or more of:
a medical volume segmentations point cloud data set.

9. The computer-implemented method of claim 1 wherein the graphical processing unit includes one or more of:
a cloud-based graphical processing unit; and
a local graphical processing unit.

10. The computer-implemented method of claim 1 wherein the three-dimensional image data set includes:
a three-dimensional image data set generated via one or more of a MRI system and CAT system.

11. A computer program product residing on a computer readable medium having a plurality of instructions stored thereon which, when executed by a processor, cause the processor to perform operations comprising:
receiving a three-dimensional image data set for processing on a medical imaging system;
processing the three-dimensional image data set on a central processing unit to generate a CPU output including one or more of:
confirming that the three-dimensional image data set is compliant with a DICOM standard,
enabling a user to define one or more of a rendering technique and rendering parameters, and
normalizing the three-dimensional image data set;
processing the three-dimensional image data set on a graphical processing unit, if the graphical processing unit is available, to calculate a segmentation volume surface normal and generate a GPU output; and
combining the CPU output and the GPU output to generate a rendered three-dimensional medical volume that depicts various tissue densities;
wherein the GPU output includes a plurality of rendered frames

12. The computer program product of claim 11 wherein processing the three-dimensional image data set on a graphical processing unit to calculate a segmentation volume surface normal and generate a GPU output includes:
processing the three-dimensional image data set on the graphical processing unit using a compute shader process to generate the GPU output.

13. The computer program product of claim 11 wherein processing the three-dimensional image data set on a graphical processing unit to calculate a segmentation volume surface normal and generate a GPU output includes:
processing the three-dimensional image data set on the graphical processing unit using a backward ray marching process to generate the GPU output.

14. The computer program product of claim 11 wherein the three-dimensional image data set includes:
a medical volume point cloud data set.

15. The computer program product of claim 11 wherein the three-dimensional image data set includes one or more of:
a medical volume segmentations point cloud data set.

16. The computer program product of claim 11 wherein the graphical processing unit includes one or more of:
a cloud-based graphical processing unit; and
a local graphical processing unit.

17. The computer program product of claim 11 wherein the three-dimensional image data set includes:
a three-dimensional image data set generated via one or more of a MRI system and CAT system.

18. A computing system including a processor and memory configured to perform operations comprising:
receiving a three-dimensional image data set for processing on a medical imaging system;
processing the three-dimensional image data set on a central processing unit to generate a CPU output;
processing the three-dimensional image data set on a graphical processing unit, if the graphical processing unit is available, to calculate a segmentation volume surface normal and generate a GPU output including one or more of:
processing the three-dimensional image data set on the graphical processing unit using a compute shader process to generate the GPU output, and
processing the three-dimensional image data set on the graphical processing unit using a backward ray marching process to generate the GPU output; and
combining the CPU output and the GPU output to generate a rendered three-dimensional medical volume that depicts various tissue densities;
wherein the GPU output includes a plurality of rendered frames.

19. The computing system of claim 18 wherein the three-dimensional image data set includes:
a medical volume point cloud data set.

20. The computing system of claim 18 wherein the three-dimensional image data set includes one or more of:
a medical volume segmentations point cloud data set.
